(19) United States
(12) Patent Application Publication
(10) Pub. No.: US 2005/0038489 A1
(43) Pub. Date: Feb. 17, 2005

(54) Electrode array for use in medical stimulation and methods thereof

(76) Inventor: Warren M. Grill, Cleveland Heights, OH (US)

Correspondence Address: Nixon Peabody LLP, Clinton Square, P.O. Box 31051, Rochester, NY, 14603-1051, US

(21) Appl. No.: 10/641,188

(22) Filed: Aug. 14, 2003

(51) Int. Cl. A61N 1/05

(52) U.S. Cl. 607/116; 607/117; 607/119

(57) ABSTRACT

An electrode array for use in medical stimulation includes one or more electrodes along an array body and one or more leads. Each of the electrodes has one or more conductive sections and each of the conductive sections has an outer surface which is substantially exposed from the array body for coupling to tissue. At least one of the electrodes has at least one of: adjacent pairs of the conductive sections separated by an insulating section; the conductive section having at least one portion which is spaced in from other portions of the conductive section and which substantially extends all the way around or all along a length of the conductive section; at least one substantially non-planar end; and a substantially planar shape with a substantially non-linear outer edge. Each of the electrodes has at least one of the leads coupled to each of the conductive sections of the electrode.

This invention was made with Government support under Grant No. R01 NS40894, 9/30/00-9/30/05, awarded by the National Institutes of Health. The Government has certain rights in the inventions.

### FIELD OF THE INVENTION

This invention relates generally to electrodes and, more particularly to an electrode array for use in medical stimulation, such as cardiac or neural stimulation.

### BACKGROUND OF THE INVENTION

A variety of different types of medical devices, such as cardiac pacemakers, defibrillators, and neural stimulators, operate on battery power. Although these medical devices are quite effective, their usefulness is limited by the life span of the batteries used to power them. One of the largest drains of power from these batteries is at the interface between an electrode in the medical device and tissue.

A circuit diagram illustrating the power drain in a medical device is illustrated in FIG. 1. A pair of leads represented by R_{leads} are coupled between a current source represented by iₛₜᵢₘ and tissue represented by Rₜᵢₛₛᵤₑ. The impedance at the interface between each of the leads R_{leads} and the tissue Rₜᵢₛₛᵤₑ is represented by Z_{interfaces}. The power consumption in this circuit is the current squared*total impedance=i\hat{ }2*(2*Rlead+2*Zinterface+Ztissue). Typically, the impedance at each of the lead-tissue interfaces Z_{interface} is much greater than the resistance for the leads R_{lead} plus the resistance of the tissue Rₜᵢₛₛᵤₑ. As a result, the power consumed in this circuit is dominated by the impedance at the electrode-tissue interfaces Z_{totalinterface}.

To reduce impedance and thereby reduce power consumption, prior systems and methods have relied on new materials and/or coatings on the electrodes. Although some of these new materials and/or coatings may reduce impedance, they also require substantial preclinical and clinical testing and large regulatory burdens before implementation.

### SUMMARY OF THE INVENTION

An electrode array for use in medical stimulation in accordance with embodiments of the present invention includes one or more electrodes along an array body and one or more leads. Each of the electrodes has one or more conductive sections and each of the conductive sections has an outer surface which is substantially exposed from the array body for coupling to tissue. At least one of the electrodes has at least one of: adjacent pairs of the conductive sections separated by an insulating section; the conductive section having at least one portion which is spaced in from other portions of the conductive section and which substantially extends all the way around or all along a length of the conductive section; at least one substantially non-planar end; and a substantially planar shape with a substantially non-linear outer edge. Each of the electrodes has at least one of the leads coupled to each of the conductive sections of the electrode.

A method for making an electrode array for use in medical stimulation in accordance with embodiments of the present invention includes providing one or more electrodes along an array body. Each of the electrodes has one or more conductive sections and each of the conductive sections has an outer surface which is substantially exposed from the array body for coupling to tissue. At least one of the electrodes has at least one of: adjacent pairs of the conductive sections separated by an insulating section; the conductive section having at least one portion which is spaced in from other portions of the conductive section and which substantially extends all the way around or all along a length of the conductive section; at least one substantially non-planar end; and a substantially planar shape with a substantially non-linear outer edge. At least one lead is coupled to each of the conductive sections of the electrode.

A method for providing medical stimulation in accordance with embodiments of the present invention includes coupling an electrode array comprising one or more electrodes along an array body to tissue. Each of the electrodes having one or more conductive sections. At least one of the electrodes has at least one of: adjacent pairs of the conductive sections separated by an insulating section; the conductive section having at least one portion which is spaced in from other portions of the conductive section and which substantially extends all the way around or all along a length of the conductive section; at least one substantially non-planar end; and a substantially planar shape with a substantially non-linear outer edge. One or more electrical pulses are applied to each of the electrodes.

An electrode array for use in medical stimulation in accordance with embodiments of the present invention includes one or more electrodes along an array body and one or more leads. Each of the electrodes has one or more conductive sections and each of the conductive sections has an outer surface which is substantially exposed from the array body for coupling to tissue. At least one of the electrodes has adjacent pairs of the conductive sections separated by an insulating section. Each of the electrodes has at least one of the leads coupled to each of the conductive sections of the electrode.

A method for making an electrode array for use in medical stimulation in accordance with embodiments of the present invention includes providing one or more electrodes along an array body. Each of the electrodes has one or more conductive sections and each of the conductive sections has an outer surface which is substantially exposed from the array body for coupling to tissue. At least one of the electrodes has adjacent pairs of the conductive sections separated by an insulating section. At least one lead is coupled to each of the conductive sections of the electrode.

A method for providing medical stimulation in accordance with embodiments of the present invention includes coupling an electrode array comprising one or more electrodes along an array body to tissue. Each of the electrodes having one or more conductive sections. At least one of the electrodes has adjacent pairs of the conductive sections separated by an insulating section. One or more electrical pulses are applied to each of the electrodes.

An electrode array for use in medical stimulation in accordance with embodiments of the present invention includes one or more electrodes along an array body and one or more leads. Each of the electrodes has one or more conductive sections and each of the conductive sections has an outer surface which is substantially exposed from the array body for coupling to tissue. At least one of the electrodes has one of the conductive sections with at least one portion which is spaced in from other portions of the conductive section and which substantially extends all the way around or all along a length of the conductive section. Each of the electrodes has at least one of the leads coupled to each of the conductive sections of the electrode.

A method for making an electrode array for use in medical stimulation in accordance with embodiments of the present invention includes providing one or more electrodes along an array body. Each of the electrodes has one or more conductive sections and each of the conductive sections has an outer surface which is substantially exposed from the array body for coupling to tissue. At least one of the electrodes has one of the conductive sections with at least one portion which is spaced in from other portions of the conductive section and which substantially extends all the way around or all along a length of the conductive section. At least one lead is coupled to each of the conductive sections of the electrode.

A method for providing medical stimulation in accordance with embodiments of the present invention includes coupling an electrode array comprising one or more electrodes along an array body to tissue. Each of the electrodes having one or more conductive sections. At least one of the electrodes has one of the conductive sections with at least one portion which is spaced in from other portions of the conductive section and which substantially extends all the way around or all along a length of the conductive section. One or more electrical pulses are applied to each of the electrodes.

An electrode array for use in medical stimulation in accordance with embodiments of the present invention includes one or more electrodes along an array body and one or more leads. Each of the electrodes has one or more conductive sections and each of the conductive sections has an outer surface which is substantially exposed from the array body for coupling to tissue. At least one of the electrodes has at least one substantially non-planar end. Each of the electrodes has at least one of the leads coupled to each of the conductive sections of the electrode.

A method for making an electrode array for use in medical stimulation in accordance with embodiments of the present invention includes providing one or more electrodes along an array body. Each of the electrodes has one or more conductive sections and each of the conductive sections has an outer surface which is substantially exposed from the array body for coupling to tissue. At least one of the electrodes has at least one substantially non-planar end. At least one lead is coupled to each of the conductive sections of the electrode.

A method for providing medical stimulation in accordance with embodiments of the present invention includes coupling an electrode array comprising one or more electrodes along an array body to tissue. Each of the electrodes having one or more conductive sections. At least one of the electrodes has at least one substantially non-planar end. One or more electrical pulses are applied to each of the electrodes.

An electrode array for use in medical stimulation in accordance with embodiments of the present invention includes one or more electrodes along an array body and one or more leads. Each of the electrodes has one or more conductive sections and each of the conductive sections has an outer surface which is substantially exposed from the array body for coupling to tissue. At least one of the electrodes has a substantially planar shape with a substantially non-linear outer edge. Each of the electrodes has at least one of the leads coupled to each of the conductive sections of the electrode.

A method for making an electrode array for use in medical stimulation in accordance with embodiments of the present invention includes providing one or more electrodes along an array body. Each of the electrodes has one or more conductive sections and each of the conductive sections has an outer surface which is substantially exposed from the array body for coupling to tissue. At least one of the electrodes has a substantially planar shape with a substantially non-linear outer edge. At least one lead is coupled to each of the conductive sections of the electrode.

A method for providing medical stimulation in accordance with embodiments of the present invention includes coupling an electrode array comprising one or more electrodes along an array body to tissue. Each of the electrodes having one or more conductive sections. At least one of the electrodes has a substantially planar shape with a substantially non-linear outer edge. One or more electrical pulses are applied to each of the electrodes.

The present invention provides an electrode array for use in medical stimulation, such as cardiac or neural stimulation, which reduces impedance and thus power consumption and thereby increases battery life. The present invention is able to reduce impedance by simply increasing the perimeter or edges of conductive sections of the electrode.

With the present invention, there is no need for the use of any exotic materials or coatings to achieve a reduction in impedance at the electrode tissue interface. This is a significant advantage because any change in the material used in an electrode array would require substantial preclinical and clinical testing and large regulatory burdens before implementation.

### BRIEF DESCRIPTION OF THE DRAWINGS

FIG. 1 is a circuit diagram of a medical device coupled to tissue;

FIG. 2 is a block diagram of system with a medical device with an electrode array in accordance with embodiments of the present invention;

FIG. 3A is a perspective view of an electrode with a segmented conductive, outer perimeter for use in an electrode array in accordance with embodiments of the present invention;

FIG. 3B is a side view of the electrode with a segmented conductive, outer perimeter shown in FIG. 3A;

FIG. 3C is a cross sectional view of an insulating section of the electrode with a segmented conductive, outer perimeter shown in FIG. 3A;

FIG. 3D is a cross sectional view of a conductive section of the electrode with a segmented conductive, outer perimeter shown in FIG. 3A

FIG. 4A is a perspective view of an electrode with a dumbbell shaped, outer perimeter for use in an electrode array in accordance with embodiments of the present invention;

FIG. 4B is a side view of the electrode with a dumbbell shaped, outer perimeter shown in FIG. 4A;

FIG. 5A is a perspective view of an electrode with substantially non-planar, serpentine-shaped ends for use in an electrode array in accordance with embodiments of the present invention;

FIG. 5B is a side view of the electrode with substantially non-planar, serpentine-shaped ends shown in FIG. 5A;

FIG. 6A is a perspective view of an electrode with conductive sections which have substantially non-planar, serpentine-shaped ends and which are separated by insulating sections for use in an electrode array in accordance with embodiments of the present invention;

FIG. 6B is a side view of the electrode with conductive sections which have substantially non-planar, serpentine-shaped ends and which are separated by insulating sections shown in FIG. 6A;

FIG. 7A is a side view of an electrode with a sinusoidal shaped, outer perimeter for use in an electrode array in accordance with embodiments of the present invention;

FIG. 7B is an end view of the electrode with a sinusoidal shaped, outer perimeter shown in FIG. 7A;

FIG. 8A is a perspective view of an electrode with spaced apart grooves slots for use in an electrode array in accordance with embodiments of the present invention;

FIG. 8B is a cross sectional view of the electrode with spaced apart grooves along the length of the electrode shown in FIG. 8A;

FIG. 9A is a top view of a planar electrode with a plurality of nested, substantially circular shaped conductive sections in accordance with embodiments of the present invention;

FIG. 9B is a top view of a planar electrode with a plurality of nested, substantially rectangular shaped, conductive sections in accordance with embodiments of the present invention;

FIG. 9C is a top view of a planar electrode with a substantially non-linear outer edge with a substantially regular pattern in accordance with embodiments of the present invention;

FIG. 9D is a top view of a planar electrode with a substantially non-linear outer edge with a substantially irregular pattern in accordance with embodiments of the present invention; and

FIG. 10 is a graph of impedance v. frequency for three different electrodes.

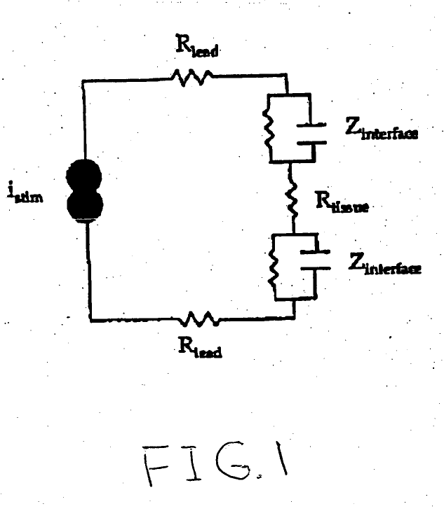

### DETAILED DESCRIPTION

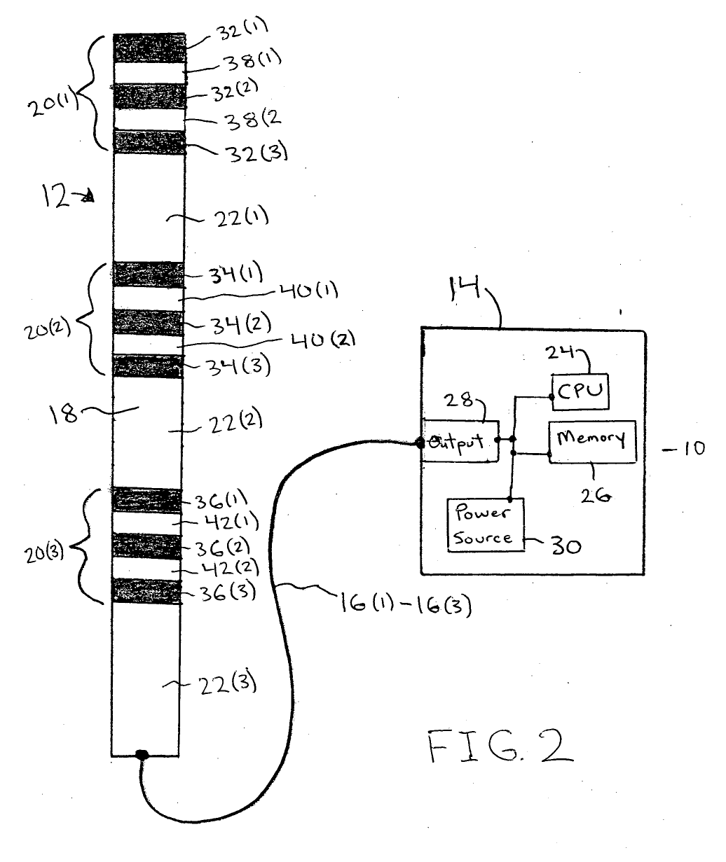

A medical device 10 with an electrode array 12 in accordance with embodiments of the present invention is illustrated in FIG. 2. The medical device 10 includes the electrode array 12 with an array body 18 and electrodes 20(1)-20(3), a pulse generator 14, and leads 16(1)-16(3), although the medical device 10 may comprise other types, numbers, and combinations of components, such as one or more of electrodes 20(4)-20(13). The present invention provides an electrode for use in an electrode array which reduces impedance and thus power consumption and thereby increases battery life by increasing the outer perimeter or edges of the electrodes in the electrode array.

Referring more specifically to FIG. 2, the electrode array 12 includes an array body 18 with electrodes 20(1)-20(3) and insulating regions 22(1)-22(3), although the electrode array 12 may comprise other types, numbers, and combinations of electrodes, insulating regions, and other components. Electrodes 20(1)-20(3) have conductive sections 32(1)-32(3), 34(1)-34(3), and 36(1)-36(3) which are each respectively separated by insulating sections 38(1)-38(2), 40(1)-40(2), and 42(1)-42(2) to form a segmented conductive, outer perimeter for each, although each of the electrodes 20(1)-20(3) may have other numbers of conductive and insulating sections. The ends of each of the conductive sections 32(1)-32(3), 34(1)-34(3), and 36(1)-36(3) are substantially planar, although other configurations for one or more of the ends of the conductive sections 32(1)-32(3), 34(1)-34(3), and 36(1)-36(3) can be used as described later herein.

The impedance of the electrodes 20(1)-20(3) with the segmented conductive outer perimeter is lower than the impedance of a prior continuous electrode. The impedance of electrodes 20(1)-20(3) decreases as the number of segments of conductive and insulating sections increases. Adding conductive sections increases the amount of edge for the electrodes 20(1)-20(3) which increases the average current density. Since impedance is inversely proportional to current density, increasing the current density decreases the impedance.

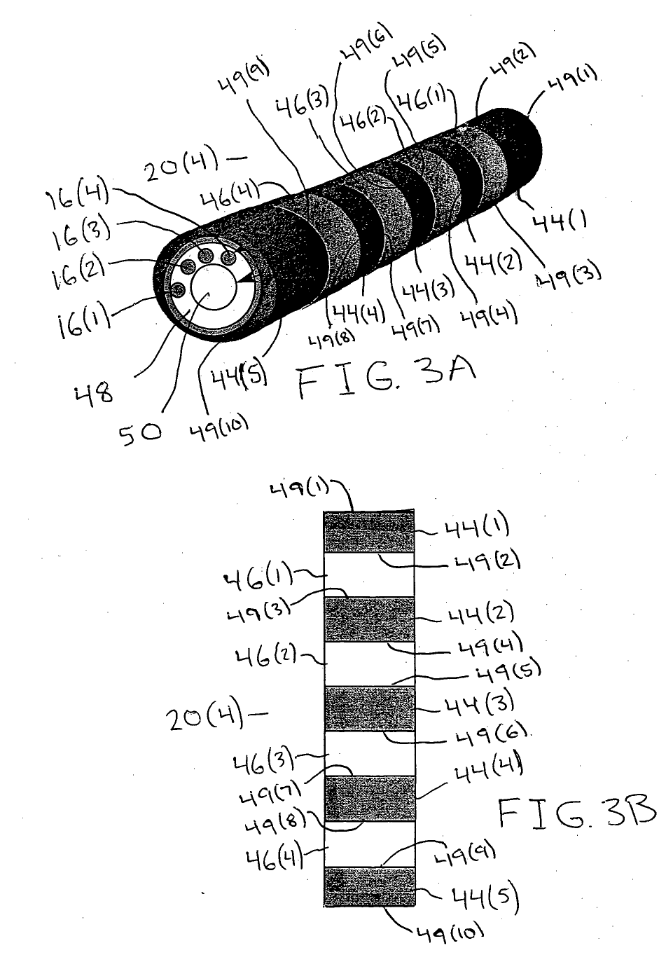

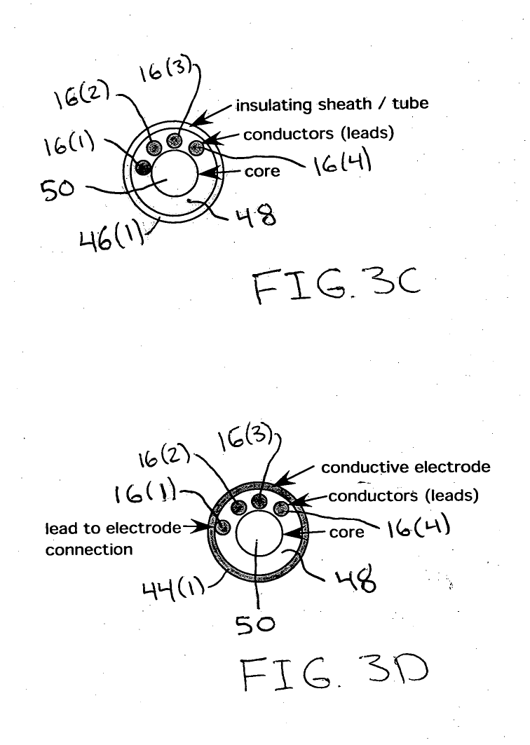

Referring to FIGS. 3A and 3B, an electrode 20(4) is illustrated which is identical to each of the electrodes 20(1)-20(3), except as described below. Elements in FIGS. 3A-3D which are identical to those described earlier have like numerals. The electrode 20(4) is interchangeable with any of the electrodes 20(1)-20(3) in the electrode array 12 as well as with any of the other electrodes 20(5)-20(13).

Electrode 20(4) has conductive sections 44(1)-44(5) which are each separated by insulating sections 46(1)-46(4) to form a segmented conductive, outer perimeters, although electrode 20(4) may have other numbers of conductive and insulating sections. The ends 49(1)-49(2), 49(3)-49(4), 49(5)-49(6), 49(7)-49(8), and 49(9)-49(10) of each of the conductive sections 44(1)-44(5) are substantially planar, although other configurations for one or more of the ends of the electrode 20(4) can be used. The impedance of the electrode 20(4) with the segmented conductive outer perimeter is lower than the impedance of each of the electrodes 20(1)-20(3) because electrode 20(4) has more segments of conductive and insulating sections. Accordingly, adding more segments of conductive sections separated by insulating sections will increase the average current density and will correspondingly decrease the impedance

Referring to FIGS. 3C-D, cross-sectional views through an insulating section 46(1) and through a conductive section 44(1) of the electrode 20(4) are illustrated. A passage 48 extends through the conductive and insulating sections 44(1) and 46(1) and through the electrode 20(4). A core 50 with leads 16(1)-16(4) are positioned in and extend along the passage 48, although other configurations with other numbers, types, and combinations of components can be used. One of the leads 16(1) is coupled to each of the conductive sections 44(1)-44(5) in the passage 48 as shown in FIG. 3D. The other leads 16(2)-16(4) are coupled to other electrodes (not shown) which are spaced along the array body 18.

Referring back to FIG. 2, the passage 48 shown in FIGS. 3C and 3D is also found, but not shown along the length of the array body 18 and extends through the conductive sections 32(1)-32(3), 34(1)-34(3), and 36(1)-36(3) and insulating sections 38(1)-38(2), 40(1)-40(2), and 42(1)-42(2) of electrodes 20(1)-20(3) and extends through insulating regions 22(1)-22(3) to form a continuous passage 48, although other configurations and numbers of passages could be used in array body 18. Lead 16(1) is coupled to the conductive sections 32(1)-32(3) of electrode 20(1), lead 16(2) is coupled to the conductive sections 34(1)-34(3) of electrode 20(2) and lead 16(3) is coupled to the conductive sections 36(1)-36(3) of electrode 20(3) via the passage 48, although other manners for making connections to the electrodes 20(1)-20(3) can be used.

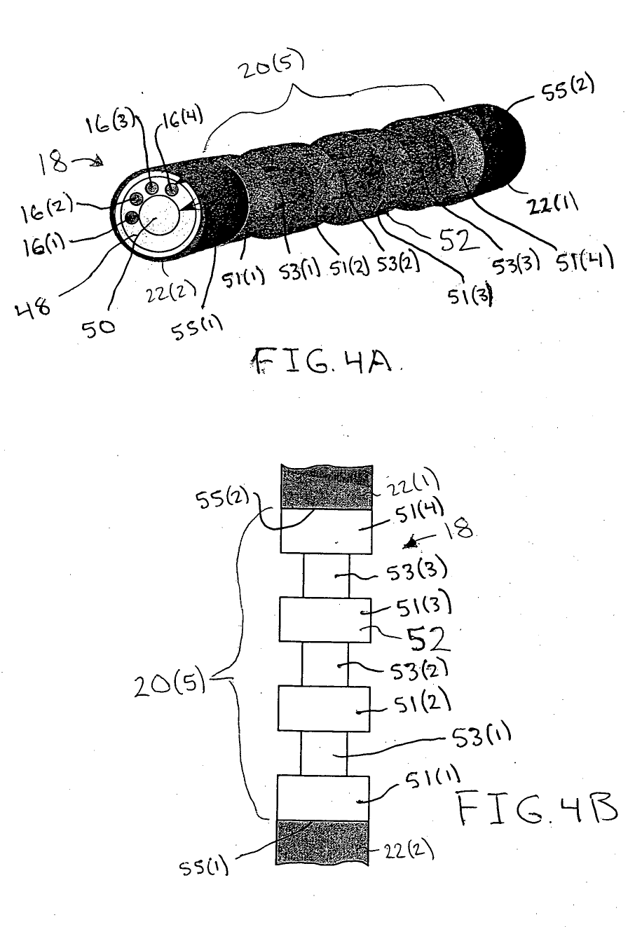

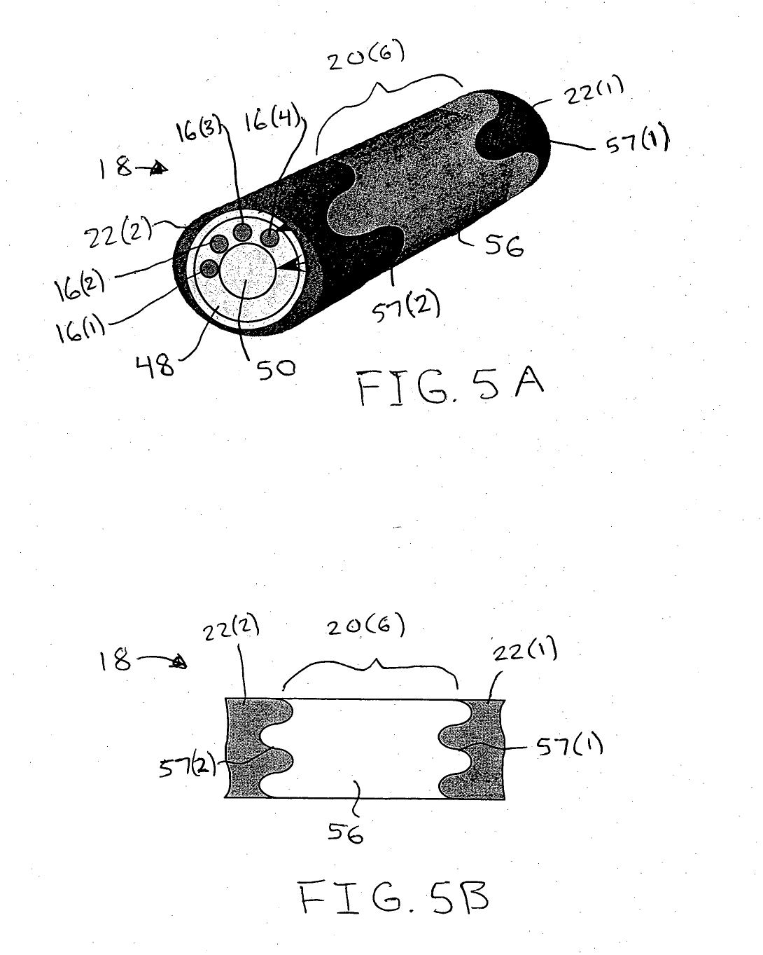

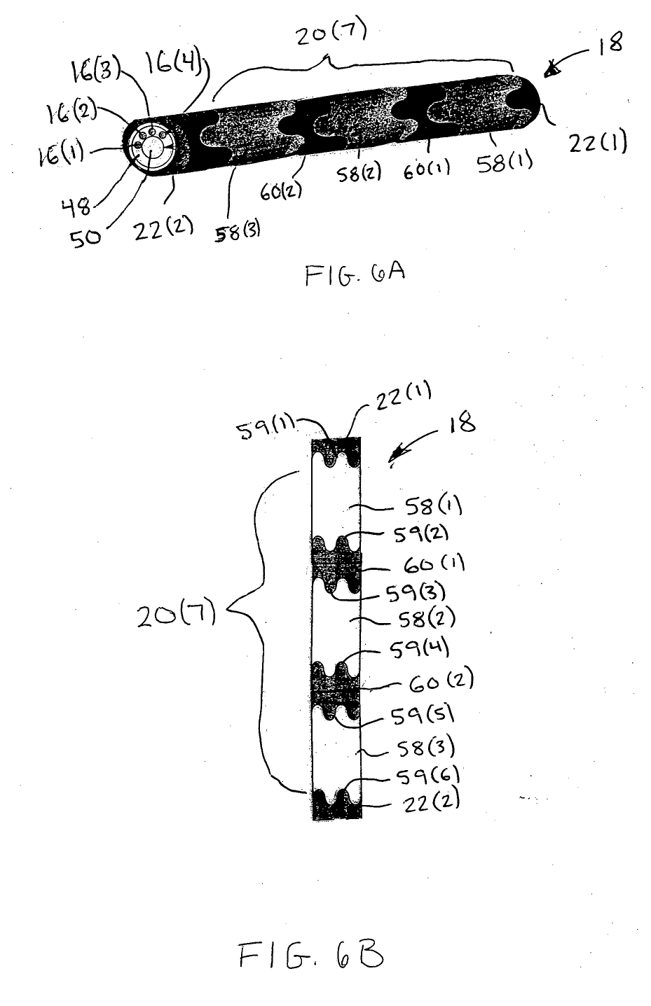

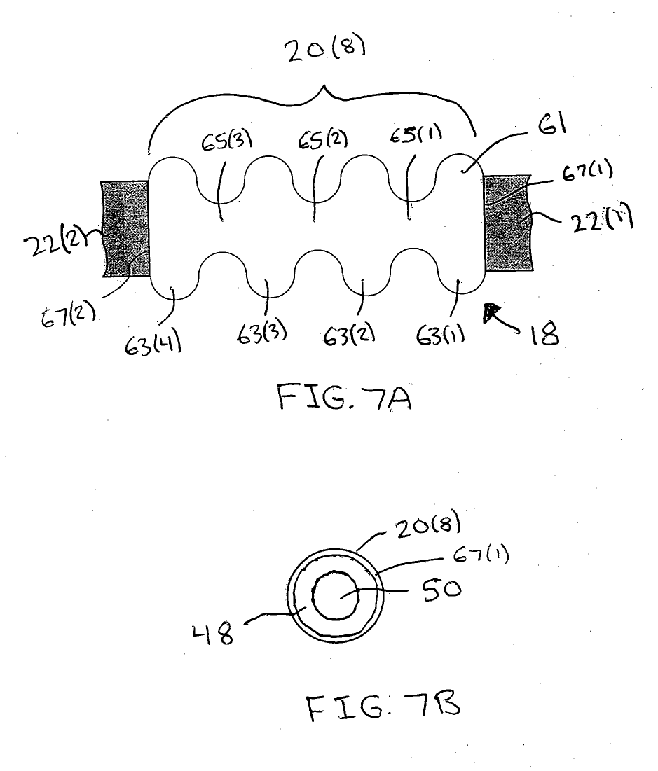

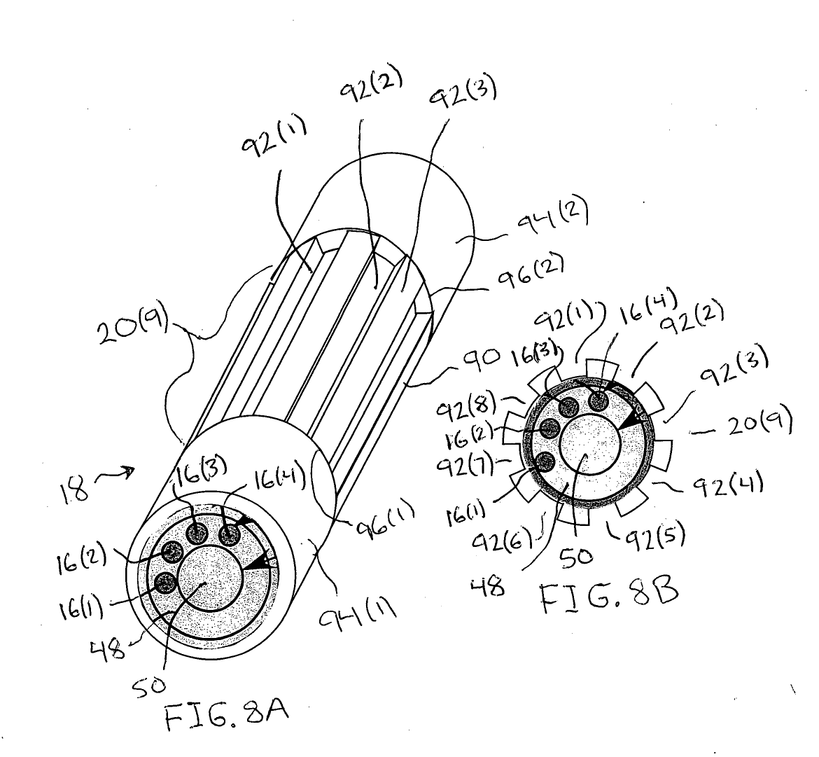

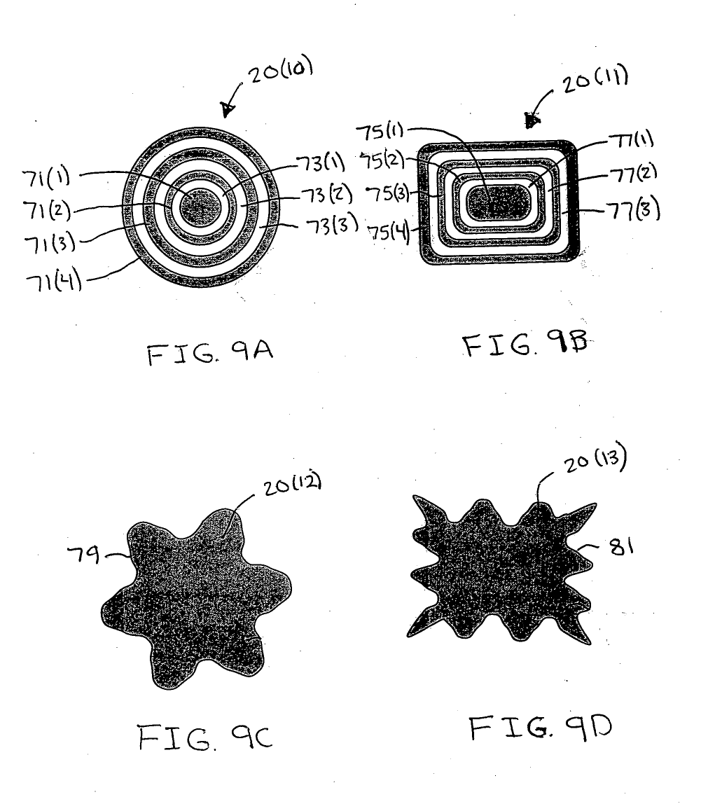

Referring to FIGS. 4A and 4B, an electrode 20(5) is illustrated which is identical to each of the electrodes 20(1)-20(3), except as described below. Elements in FIGS. 3C-3D and 4A-4B which are identical to those described earlier have like numerals. The electrode 20(5) is interchangeable with any of the electrodes 20(1)-20(3) in the electrode array 12 as well as with any of the other electrodes 20(4), and 20(6)-20(13).

Electrode 20(5) has a conductive section 52 with dumbbell end portions 51(1)-51(4) which are respectively separated by central bar portions 53(1)-53(3) to form a repeated, dumbbell-shaped, outer perimeter, although the outer perimeter of electrode 20(5) may have other non-linear shapes. The conductive section 52 which forms electrode 20(5) is located between insulating regions 22(1) and 22(2) in array body 18, although electrode 20(5) can be used in other locations in the array body 18. The ends 55(1)-55(2) of the conductive section 52 which are substantially planar, although other configurations for one or more of the ends 55(1)-55(2) of the electrode 20(5) can be used. The passage 48 with the core 50 is also found along the length of the conductive section 52 which forms the electrode 20(5).

The impedance for electrode 20(5) decreases as the number of dumbbell end portions which are each separated by a central bar portions for the conductive section 52 increases. Adding dumbbell end portions and central bar portions for the shape of the outer perimeter of the conductive section 52 which extend around the outer perimeter of the electrode 20(5) increases the amount of edge for the electrode 20(5) which increases the average current density. Since impedance is inversely proportional to current density, increasing the current density decreases the impedance. Indented or recessed portions or grooves in an electrode which do not substantially extend all the way around or all along the entire length of the outer perimeter would not maximize the potential increase in the average current density and the corresponding potential decrease in impedance.

Referring to FIGS. 5A and 5B, an electrode 20(6) is illustrated which is identical to each of the electrodes 20(1)-20(3), except as described below. Elements in FIGS. 3C-3D and 5A-5B which are identical to those described earlier have like numerals. The electrode 20(6) is interchangeable with any of the electrodes 20(1)-20(3) in the electrode array 12 as well as with any of the other electrodes 20(4)-20(5), and 20(7)-20(13).

Electrode 20(6) has a conductive section 56 which has substantially serpentine shaped ends 57(1)-57(2), although the ends 57(1)-57(2) of conductive section 56 could have other non-planar shapes. The conductive section 56 which forms electrode 20(6) is located between insulating regions 22(1) and 22(2) in array body 18, although electrode 20(6) can be used in other locations in the array body 18. The passage 48 with the core 50 is also found along the length of the conductive section 56 which forms electrode 20(6).

The impedance for electrode 20(6) decreases as the size of the edge along the non-planar ends of the conductive section 56 increases. Increasing the amount of edge along the non-planar ends of the conductive section increases the average current density. Again, since impedance is inversely proportional to current density, increasing the current density decreases the impedance.

Referring to FIGS. 6A and 6B, an electrode 20(7) is illustrated which is identical to each of the electrodes 20(1)-20(3), except as described below. Elements in FIGS. 3C-3D and 6A-6B which are identical to those described earlier have like numerals. The electrode 20(7) is interchangeable with any of the electrodes 20(1)-20(3) in the electrode array 12 as well as with any of the other electrodes 20(4)-20(6), and 20(8)-20(13).

Electrode 20(7) has conductive sections 58(1)-58(3) which each have substantially serpentine-shaped ends 59(1)-59(2), 59(3)-59(4), and 59(5)-59(6) and which are each respectively separated by insulating sections 60(1) and 60(2), although electrode 20(7) may have other shapes for ends 59(1)-59(2), 59(3)-59(4), and 59(5)-59(6) and other numbers of conductive and insulating sections. The electrode 20(7) is located between insulating regions 22(1) and 22(2) in array body 18, although other electrode 20(7) can be used in other locations in the array body 18. The passage 48 with the core 50 is also found along the length of the conductive section 52 which forms the electrode 20(7).

The impedance of the electrode 20(7) with the segmented conductive outer perimeter and with the substantially serpentine-shaped ends is lower than the impedance of the electrode 20(6) with the substantially serpentine-shaped ends because electrode 20(7) has more segments of conductive and insulating sections. The impedance of electrode 20(7) decreases as the number of segments of conductive and insulating sections increases and decreases as the size of the edge along the non-planar ends of the conductive sections 58(1)-58(3) increases. Adding conductive sections increases the amount of edge for the electrode 20(7) and adding non-planar ends also increases the amount of edge which increases the average current density. Since impedance is inversely proportional to current density, increasing the current density decreases the impedance.

Referring to FIGS. 7A and 7B, an electrode 20(8) is illustrated which is identical to electrode 20(5), except as described below. Elements in FIGS. 7A-7B which are identical to those described earlier have like numerals. The electrode 20(8) is interchangeable with any of the other electrodes 20(1)-20(3) in the electrode array 12 as well as with any of the other electrodes 20(4)-20(7) and 20(9)-20(13).

Electrode 20(8) has a conductive section 61 with extended portions 63(1)-63(4) which are respectively separated by indented portions 65(1)-65(3) to form a repeated, sinusoidal-shaped, outer perimeter, although the outer perimeter of electrode 20(5) may have a variety of other non-linear shapes. The conductive section 61 which forms electrode 20(8) is located between insulating regions 22(1) and 22(2) in array body 18, although electrode 20(8) can be used in other locations in the array body 18. The ends 67(1)-67(2) of the conductive section 61 which are substantially planar, although other configurations for one or more of the ends 67(1)-67(2) of the electrode 20(8) can be used. The passage 48 with the core 50 is also found along the length of the conductive section 52 which forms the electrode 20(5). For ease of illustration only, the leads are not shown in the passage 48 shown in FIG. 7B.

The impedance for electrode 20(8) decreases as the number of extended portions which are each separated by indented portions for the conductive section 61 increases. Adding extended portions and indented portions for the shape of the outer perimeter of the conductive section 61 increases the amount of edge for the electrode 20(8) which increases the average current density. Since impedance is inversely proportional to current density, increasing the current density decreases the impedance. Indented or recessed portions or grooves in an electrode which do not substantially extend all the way around or all along the entire length of the outer perimeter would not maximize the potential increase in the average current density and the corresponding potential decrease in impedance.

Referring to FIGS. 8A-8B, an electrode 20(9) is illustrated which is identical to each of the electrode 20(5), except as described below. Elements in FIGS. 8A-8B which are identical to those described earlier have like numerals. The electrode 20(9) is interchangeable with any of the electrodes 20(1)-20(3) in the electrode array 12 as well as with any of the other electrodes 20(4)-20(8) and 20(10)-20(13).

Electrode 20(9) has a conductive section 90 with spaced apart grooves 92(1)-92(8) which extend along the length of and around the outer perimeter of the electrode 20(9) to form a cog-shaped, cross-sectional outer perimeter, although the outer perimeter of electrode 20(9) may have other configurations, such as grooves which extend in a diagonal pattern along the length of the electrode. The conductive section 90 which forms electrode 20(9) is located between insulating regions 94(1) and 94(2) in array body 18, although electrode 20(9) can be used in other locations in the array body 18. The ends 96(1)-96(2) of the conductive section 90 are substantially planar, although other configurations for one or more of the ends 96(1)-96(2) of the electrode 20(9) can be used. The passage 48 with the core 50 is also found along the length of the conductive section 90 which forms the electrode 20(9).

The impedance for electrode 20(9) decreases as the number of grooves which extend along the length of and around the outer perimeter of the electrode increases. Adding grooves 92(1)-92(8) which extend along the length of and around the outer perimeter of the conductive section 90 of the electrode 20(9) increases the amount of edge for the electrode 20(9) which increases the average current density. Since impedance is inversely proportional to current density, increasing the current density decreases the impedance. Indented or recessed portions or grooves in an electrode which do not substantially extend all the way around or all along the entire length of the outer perimeter would not maximize the potential increase in the average current density and the corresponding potential decrease in impedance.

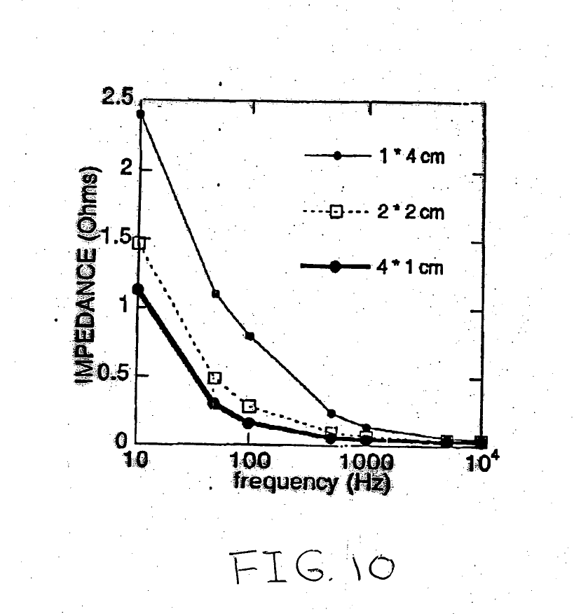

Referring to FIGS. 9A-9D, a variety of different types of electrodes 20(10)-20(13) are illustrated. Each of these electrodes 20(10)-20(13) has a substantially planar or flat shape and can be used in a variety of different applications, such as a defibrillation patch electrode. are illustrated.

The planar electrode 20(10) has a plurality of nested, substantially circular shaped conductive sections 71(4)-71(4), although the electrode 20(10) could have other shapes, such as square or rectangular. The plurality of conductive sections 71(1)-71(4) are respectively separated by insulating sections 73(1)-73(3).

The planar electrode 20(11) has a plurality of nested, substantially rectangular shaped, conductive sections 75(1)-75(4) although the electrode 20(11) could have other shapes, such as circular or square. The plurality of conductive sections 75(1)-75(4) are respectively separated by insulating sections 77(1)-77(3).

The planar electrode 20(12) has a conductive section 79 with a substantially non-linear outer edge with a substantially regular pattern, although the electrode 20(12) could have other patterns for the non-linear outer edge. The planar electrode 20(13) also has a conductive section 81 with a substantially non-linear outer edge, but with a substantially irregular pattern, although the electrode 20(13) could have other patterns for the non-linear outer edge.

The impedance for planar electrodes 20(10) and 20(11) decreases as the number of segments of conductive and insulating sections increases. Adding conductive sections increases the amount of edge for the electrodes 20(10) and 20(11) which increases the average current density. Since impedance is inversely proportional to current density, increasing the current density decreases the impedance.

The impedance for planar electrodes 20(12) and 20(13) decreases as the length of the edge along the outer perimeter of the conductive sections 79 and 81 increases. Increases the amount of edge for the electrodes 20(12) and 20(13) increases the average current density. Since impedance is inversely proportional to current density, increasing the current density decreases the impedance.

Electrodes 20(1)-20(13) have been described to illustrate different ways to alter the geometry of the electrode to reduce impedance, although other combinations of these alterations and other geometrical configurations which increase the outer perimeter or edges of the conductive sections of the electrodes can also be used.

Referring back to FIG. 2, the medical device 10 also includes a pulse generator 14 which is coupled to the electrodes 20(1)-20(3) via leads 16(1)-16(2), although other types of devices for transmitting and/or receiving pulses or signals can be used. In this particular embodiment, the pulse generator 14 includes a central processing unit (CPU) 24, a memory 26, an output device 28 and a power source 30, although the pulse generator 14 can have other components, other numbers of components, and other combinations of components which are coupled together in other manners. The memory 26 stores programmed instructions and data for delivering electrical pulses to one or more of the electrodes 20(1)-20(3) via leads 16(1)-16(3), although some or all of these instructions and data may be stored elsewhere. Since the processes for controlling and delivery electrical pulses are well known to those of ordinary skill in the art they will not be described in detail here. The output device 28 in pulse generator 14 is coupled to electrodes 20(1)-20(3) via the leads 16(1)-16(3). The power source 30 is a battery, although other types of power sources can be used.

The method for making the electrode array 12 will be described with reference to FIGS. 2, 3C, and 3D. Electrodes 20(1)-20(3) spaced along and are respectively separated by insulating regions 22(1)-22(3) along an array body 18, although other types, numbers, and combinations of electrodes and insulating regions, such as one or more of electrodes 20(4)-20(9) could be used. Leads 16(1)-16(3) are passed along passage 48 and are each coupled to one of the electrodes 20(1)-20(3), although other manners for making connections to the electrodes 20(1)-20(3) can be used. More specifically, in this particular embodiment lead 16(1) is coupled to the conductive sections 32(1)-32(3) of electrode 20(1), lead 16(2) is coupled to the conductive sections 34(1)-34(3) of electrode 20(2) and lead 16(3) is coupled to the conductive sections 36(1)-36(3) of electrode 20(3) via the passage 48. The other end of leads 16(1)-16(3) are coupled to pulse generator 14, although leads 16(1)-16(3) can be coupled to other devices.

The method for making an electrode array with one or more of the electrodes 20(4)-20(9) is identical to the method of making an electrode array 12 with electrodes 20(1)-20(3), except as described below. One or more of the electrodes 20(4)-20(9) may also be used with one or more of the electrodes 2091)-20(3). One or more of the electrodes 20(4)-20(9) are spaced along and if more than one electrode is used are respectively separated by one or more insulating regions along an array body 18, although other types, numbers, and combinations of electrodes and insulating regions could be used. A lead is passed along a passage 48 for each of the electrodes and is coupled to one of the electrodes, although other manners for making connections to the one or more electrodes can be used. More specifically, one lead would be coupled to conductive sections 44(1)-44(5) of electrode 20(4), one lead would be coupled to conductive section 52 of electrode 20(5), one lead would be coupled to conductive section 56 of electrode 20(6), one lead would be coupled to conductive sections 58(1)-58(3) of electrode 20(7), one lead would be coupled to conductive section 61 of electrode 20(8), and one lead would be coupled to conductive section 90 of electrode 20(9), depending on which of the one or more electrodes 20(4)-20(9) were used. The other end of the one or more leads are coupled to pulse generator 14, although leads could be coupled to other devices.

The method for making the electrode array with electrodes 20(10)-20(13) will be described with reference to FIGS. 9A-9D. A lead is coupled to the particular electrode 20(10), 20(11), 20(12), or 20(13), although other manners for making connections to the electrodes can be used. More specifically, the lead is coupled to the conductive sections 71(1)-71(4) of electrode 20(10), the lead is coupled to the conductive sections 75(1)-75(4) of electrode 20(11), the lead is coupled to the conductive section 79 of electrode 20(12), and the lead is coupled to the conductive section 81 of electrode 20(13). The other end of each of these leads is coupled to pulse generator 14, although leads can be coupled to other devices

The operation of a medical device 10 with an electrode array 12 the electrode in accordance with embodiments of the present invention will now be described with reference to FIGS. 2 and 3A-3D. The pulse generator 14 generates pulses which are transmitted on to one or more of the leads 16(1)-16(3) coupled to the output device 28. The leads 16(1)-16(3) are each coupled to one of the electrodes 20(1)-20(3), respectively, which transmit the pulses to adjacent tissue. With the present invention, the impedance at the interface between the electrodes 20(1)-20(3) and the adjacent tissue is decreased by increasing the outer perimeter or edge with the segments in the electrodes 20(1)-20(3) in the electrode array 12. As a result, power consumption for this medical device 10 is reduced and battery life is increased when compared against a medical device with continuous electrodes. With the electrodes 20(4)-20(13), the operation of the medical device 10 is the same, except that the pulses from the pulse generator 14 are transmitted by the leads to one or more of the other electrodes 20(4)-20(13), depending on which of the one or more electrodes are being used in the application.

The present invention recognized that current density on an electrode is not distributed uniformly across the surface. Rather, current density J is very much higher at the edges of the electrode than near the center of the electrode. Accordingly, as described earlier the present invention takes advantage of this by increasing the amount of edge for the electrodes 20(1)-20(13) which increases the average current density. Since impedance is inversely proportional to current density, increasing the current density decreases the impedance.

An experiment illustrating the feasibility of the present invention was conducted for three electrodes of equal conductive area. More specifically, the impedance of an electrode with a single continuous conductor having a length of four centimeters represented by (1*4 cm) in FIG. 10, the impedance of an electrode with two conductive segments each having a length of two centimeters represented by (2*2 cm) in FIG. 10, and the impedance of an electrode with four conductive segments each having a length of one centimeter represented by (4*1 cm) in FIG. 10 as a function of frequency were tested.

As illustrated in the graph shown in FIG. 10, the impedance of the electrodes with segments (2*2 cm and 4*1 cm) was lower than that of the electrode with the single continuous conductor (1*4 cm). Additionally, the impedance of the electrodes with four conductive segments (4*1 cm) was lower than that of the electrode with two conductive segments (2*2 cm) Accordingly, as this graph illustrates the impedance decreases as the number of conductive segments or the outer perimeter of the electrodes increases.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefor, is not intended to limit the claimed processes to any order except as may be specified in the claims. Accordingly, the invention is limited only by the following claims and equivalents thereto.

1. An electrode array for use in medical stimulation, the array comprising:
an array body;
one or more electrodes along the array body, each of the electrodes having one or more conductive sections, each of the conductive sections having an outer surface which is substantially exposed from the array body for coupling to tissue, and at least one of the electrodes having at least one of: adjacent pairs of the conductive sections separated by an insulating section; one of the conductive sections with at least one portion which is spaced in from other portions of the conductive section and which substantially extends all the way around or all along a length of the conductive section; at least one substantially non-planar end; and a substantially planar shape with a substantially non-linear outer edge; and
one or more leads, wherein each of the electrodes has at least one of the leads coupled to each of the conductive sections of the electrode.

2. The array as set forth in claim 1 wherein an end of one of the conductive sections adjacent one of the insulating sections is substantially planar.

3. The array as set forth in claim 1 wherein an end of one of the conductive sections adjacent one of the insulating sections is substantially non-planar.

4. The array as set forth in claim 3 wherein the substantially non-planar end has a substantially serpentine shape.

5. The array as set forth in claim 1 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

6. The array as set forth in claim 1 wherein the substantially non-planar end has a substantially serpentine shape.

7. The array as set forth in claim 1 wherein the substantially planar shape with a substantially non-linear outer edge has a regular pattern.

8. The array as set forth in claim 1 wherein the substantially planar shape with a substantially non-linear outer edge has an irregular pattern.

9. A method for making an electrode array for use in medical stimulation, the method comprising:
providing one or more electrodes along an array body, each of the electrodes having one or more conductive sections, each of the conductive sections having an outer surface which is substantially exposed from the array body for coupling to tissue, and at least one of the electrodes having at least one of: adjacent pairs of the conductive sections separated by an insulating section; one of the conductive sections with at least one portion which is spaced in from other portions of the conductive section and which substantially extends all the way around or all along a length of the conductive section; at least one substantially non-planar end; and a substantially planar shape with a substantially non-linear outer edge; and
coupling at least one lead to each of the conductive sections of the electrode.

10. The method as set forth in claim 9 wherein an end of one of the conductive sections adjacent one of the insulating sections is substantially planar.

11. The method as set forth in claim 9 wherein an end of one of the conductive sections adjacent one of the insulating sections is substantially non-planar.

12. The method as set forth in claim 11 wherein the substantially non-planar end has a substantially serpentine shape.

13. The method as set forth in claim 9 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

14. The method as set forth in claim 9 wherein the substantially non-planar end has a substantially serpentine shape.

15. The method as set forth in claim 9 wherein the substantially planar shape with a substantially non-linear outer edge has a regular pattern.

16. The method as set forth in claim 9 wherein the substantially planar shape with a substantially non-linear outer edge has an irregular pattern.

17. A method for providing medical stimulation, the method comprising:
coupling an electrode array comprising one or more electrodes along an array body to tissue, each of the electrodes having one or more conductive sections, at least one of the electrodes having at least one of: adjacent pairs of the conductive sections separated by an insulating section; one of the conductive sections with at least one portion which is spaced in from other portions of the conductive section and which substantially extends all the way around or all along a length of the conductive section; at least one substantially non-planar end; and a substantially planar shape with a substantially non-linear outer edge; and
applying one or more electrical pulses to each of the electrodes.

18. The method as set forth in claim 17 wherein an end of one of the conductive sections adjacent one of the insulating sections is substantially planar.

19. The method as set forth in claim 17 wherein an end of one of the conductive sections adjacent one of the insulating sections is substantially non-planar.

20. The method as set forth in claim 19 wherein the substantially non-planar end has a substantially serpentine shape.

21. The method as set forth in claim 17 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

22. The method as set forth in claim 17 wherein the substantially non-planar end has a substantially serpentine shape.

23. The method as set forth in claim 17 wherein the substantially planar shape with a substantially non-linear outer edge has a regular pattern.

24. The method as set forth in claim 17 wherein the substantially planar shape with a substantially non-linear outer edge has an irregular pattern.

25. An electrode array for use in medical stimulation, the array comprising:
an array body;
one or more electrodes along the array body, each of the electrodes having one or more conductive sections, each of the conductive sections having an outer surface which is substantially exposed from the array body for coupling to tissue, and at least one of the electrodes having adjacent pairs of the conductive sections separated by an insulating section; and
one or more leads, wherein each of the electrodes has at least one of the leads coupled to each of the conductive sections of the electrode.

26. The array as set forth in claim 25 wherein an end of one of the conductive sections adjacent one of the insulating sections is substantially planar.

27. The array as set forth in claim 25 wherein an end of one of the conductive sections adjacent one of the insulating sections is substantially non-planar.

28. The array as set forth in claim 27 wherein the substantially non-planar end has a substantially serpentine shape.

29. A method for making an electrode array for use in medical stimulation, the method comprising:
providing one or more electrodes along an array body, each of the electrodes having one or more conductive sections, each of the conductive sections having an outer surface which is substantially exposed from the array body for coupling to tissue, and at least one of the electrodes having adjacent pairs of the conductive sections separated by an insulating section; and
coupling at least one lead to each of the conductive sections of the electrode.

30. The method as set forth in claim 29 wherein an end of one of the conductive sections adjacent one of the insulating sections is substantially planar.

31. The method as set forth in claim 29 wherein an end of one of the conductive sections adjacent one of the insulating sections is substantially non-planar.

32. The method as set forth in claim 31 wherein the substantially non-planar end has a substantially serpentine shape.

33. A method for providing medical stimulation, the method comprising:
coupling an electrode array comprising one or more electrodes along an array body to tissue, each of the electrodes having one or more conductive sections, at least one of the electrodes having adjacent pairs of the conductive sections separated by an insulating section; and
applying one or more electrical pulses to each of the electrodes.

34. The method as set forth in claim 33 wherein an end of one of the conductive sections adjacent one of the insulating sections is substantially planar.

35. The method as set forth in claim 33 wherein an end of one of the conductive sections adjacent one of the insulating sections is substantially non-planar.

36. The method as set forth in claim 35 wherein the substantially non-planar end has a substantially serpentine shape.

37. An electrode array for use in medical stimulation, the array comprising:
an array body;
one or more electrodes along the array body, each of the electrodes having one or more conductive sections, each of the conductive sections having an outer surface which is substantially exposed from the array body for coupling to tissue, and at least one of the electrodes having one of the conductive sections with at least one portion which is spaced in from other portions of the conductive section and which substantially extends all the way around or all along a length of the conductive section;
one or more leads, wherein each of the electrodes has at least one of the leads coupled to each of the conductive sections of the electrode.

38. The array as set forth in claim 37 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

39. A method for making an electrode array for use in medical stimulation, the method comprising:
providing one or more electrodes along an array body, each of the electrodes having one or more conductive sections, each of the conductive sections having an outer surface which is substantially exposed from the array body for coupling to tissue, and at least one of the electrodes having one of the conductive sections with at least one portion which is spaced in from other portions of the conductive section and which substantially extends all the way around or all along a length of the conductive section; and
coupling at least one lead to each of the conductive sections of the electrode.

40. The method as set forth in claim 39 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

41. A method for providing medical stimulation, the method comprising:
coupling an electrode array comprising one or more electrodes along an array body to tissue, each of the electrodes having one or more conductive sections, at least one of the electrodes having one of the conductive sections with at least one portion which is spaced in from other portions of the conductive section and which substantially extends all the way around or all along a length of the conductive section; and
applying one or more electrical pulses to each of the electrodes.

42. The method as set forth in claim 41 wherein the conductive section having at least one portion which is spaced in from other portions of the conductive section and which substantially extends all the way around or all along an entire length of the conductive section is at least one of dumbbell shaped, sinusoidal shaped, and cog cross-sectional shape.

43. An electrode array for use in medical stimulation, the array comprising:
an array body;
one or more electrodes along the array body, each of the electrodes having one or more conductive sections, each of the conductive sections having an outer surface which is substantially exposed from the array body for coupling to tissue, and at least one of the electrodes having at least one substantially non-planar end; and
one or more leads, wherein each of the electrodes has at least one of the leads coupled to each of the conductive sections of the electrode.

44. The array as set forth in claim 43 wherein the substantially non-planar end has a substantially serpentine shape.

45. A method for making an electrode array for use in medical stimulation, the method comprising:
providing one or more electrodes along an array body, each of the electrodes having one or more conductive sections, each of the conductive sections having an outer surface which is substantially exposed from the array body for coupling to tissue, and at least one of the electrodes having at least one substantially non-planar end; and
coupling at least one lead to each of the conductive sections of the electrode.

46. The method as set forth in claim 45 wherein the substantially non-planar end has a substantially serpentine shape.

47. A method for providing medical stimulation, the method comprising:
coupling an electrode array comprising one or more electrodes along an array body to tissue, each of the electrodes having one or more conductive sections, at least one of the electrodes having at least one substantially non-planar end; and
applying one or more electrical pulses to each of the electrodes.

48. The method as set forth in claim 47 wherein the substantially non-planar end has a substantially serpentine shape.

49. An electrode array for use in medical stimulation, the array comprising:
an array body;
one or more electrodes along the array body, each of the electrodes having one or more conductive sections, each of the conductive sections having an outer surface which is substantially exposed from the array body for coupling to tissue, and at least one of the electrodes having a substantially planar shape with a substantially non-linear outer edge; and
one or more leads, wherein each of the electrodes has at least one of the leads coupled to each of the conductive sections of the electrode.

50. The array as set forth in claim 49 wherein the substantially non-linear outer edge has a regular pattern.

51. The array as set forth in claim 49 wherein the substantially non-linear outer edge has an irregular pattern.

52. A method for making an electrode array for use in medical stimulation, the method comprising:
providing one or more electrodes along an array body, each of the electrodes having one or more conductive sections, each of the conductive sections having an outer surface which is substantially exposed from the array body for coupling to tissue, and at least one of the electrodes having a substantially planar shape with a substantially non-linear outer edge; and
coupling at least one lead to each of the conductive sections of the electrode.

53. The method as set forth in claim 52 wherein the substantially non-linear outer edge has a regular pattern.

54. The method as set forth in claim 52 wherein the substantially non-linear outer edge has an irregular pattern.

55. A method for providing medical stimulation, the method comprising:
coupling an electrode array comprising one or more electrodes along an array body to tissue, each of the electrodes having one or more conductive sections, at least one of the electrodes having a substantially planar shape with a substantially non-linear outer edge; and
applying one or more electrical pulses to each of the electrodes.

56. The method as set forth in claim 55 wherein the substantially non-linear outer edge has a regular pattern.

57. The method as set forth in claim 55 wherein the substantially non-linear outer edge has an irregular pattern.
